(19) United States
(12) Patent Application Publication
(10) Pub. No.: US 2007/0038027 A1
(43) Pub. Date: Feb. 15, 2007

(54) Endoscope hole sealing part

(75) Inventors: Masaaki Miyagi, Tokyo (JP); Seisuke Takase, Tokyo (JP); Hidenobu Kimura, Tokyo (JP)

Correspondence Address: Thomas Spinelli;Scully, Scott, Murphy & Presser, 400 Garden City Plaza, Garden City, NY, 11530, US

(73) Assignee: Olympus Medical Systems Corp., Tokyo (JP)

(21) Appl. No.: 11/200,402

(22) Filed: Aug. 9, 2005

(51) Int. Cl. A61B 1/04; A61B 1/00

(52) U.S. Cl. 600/130; 600/129; 600/128; 600/127

(57) ABSTRACT

An endoscope hole sealing part including a body portion which seals a screw hole into which a fixing member is inserted, the fixing member fixing a built-in part in an endoscope, and an end face which is one surface of the body portion and is the outer surface of the endoscope.

### BACKGROUND OF THE INVENTION

1. Field of the Invention

The present invention relates to an endoscope hole sealing part for use in an endoscope having an air/water feeding nozzle.

2. Description of the Related Art

An endoscope apparatus has been known by which the inside of a body cavity of a subject can be observed. The endoscope apparatus includes an insertion portion to be inserted into a body cavity of a subject and an operation portion disposed on the proximal side of the insertion portion.

In order to observe the inside of a body cavity of a subject, the distal side of the insertion portion of the endoscope apparatus may be inserted into the body cavity through the mouth or the anus, for example, of the subject, and the image of an observed part can be shot by an image pickup unit disposed on the distal end of the insertion portion. After the image of the shot part is conducted to the operation portion through a signal line disposed within the insertion portion, the image is displayed on an external monitor through a universal cord.

The insertion portion of the endoscope has the distal end section including a rigid member at the distal end part thereof. The distal surface of the distal end section has an objective lens of the image pickup unit and an air/water feeding nozzle for spraying cleaning water or cleaning air to the objective lens through a conduit within the insertion portion. The surface of the objective lens can be cleaned by cleaning water or cleaning air sprayed to the objective lens from the air/water feeding nozzle so that clear images of the inside of a body cavity can be always obtained.

For example, Japanese Unexamined Patent Application Publication No. 06-237889 discloses such an air/water feeding nozzle. The air/water feeding nozzle is fixed with a fixing member at the distal end part of the insertion portion. The fixing member is inserted into a screw hole, which is a hole in the distal end part of the insertion portion and fixes the air/water feeding nozzle at the distal end part.

### SUMMARY OF THE INVENTION

An endoscope hole sealing part includes a body portion which seals a hole into which a fixing member is inserted, the fixing member fixing a built-in part in an endoscope, and an end face which is one surface of the body portion and is the outer surface of the endoscope.

The above and other objects, features and advantages of the invention will become more clearly understood from the following description referring to the accompanying drawings.

### BRIEF DESCRIPTION OF THE DRAWINGS

FIG. 1 is an explanatory diagram schematically showing an endoscope apparatus according to an embodiment of the invention;

FIG. 2 is a plan view showing the distal surface of an insertion portion of the endoscope;

FIG. 3 is a section diagram of the distal end section of the endoscope taken along the Line III-III in FIG. 2;

FIG. 4 is a partial section diagram of the distal end section of the endoscope taken along the Line IV-IV in FIG. 3;

FIG. 5 is an appearance diagram of a sealing pin, which is an endoscope hole sealing part of the invention;

FIG. 6 is a diagram for explaining assembly in which an air/water feeding nozzle is inserted from the distal surface of a distal cover to a distal rigid part;

FIG. 7 is a diagram for explaining assembly in which a fixing member is inserted from a hole in the distal cover to the distal rigid part;

FIG. 8 is a diagram for explaining assembly in which the sealing pin is inserted into the hole in the distal cover; and FIG. 9 is a perspective view of the distal end section in the state shown in FIG. 8.

### DESCRIPTION OF THE PREFERRED EMBODIMENTS

Embodiments of the invention will be described below with reference to drawings.

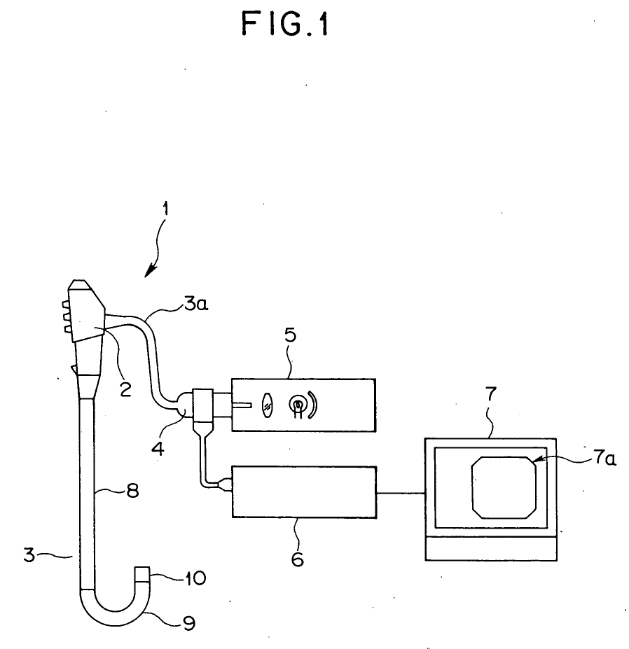

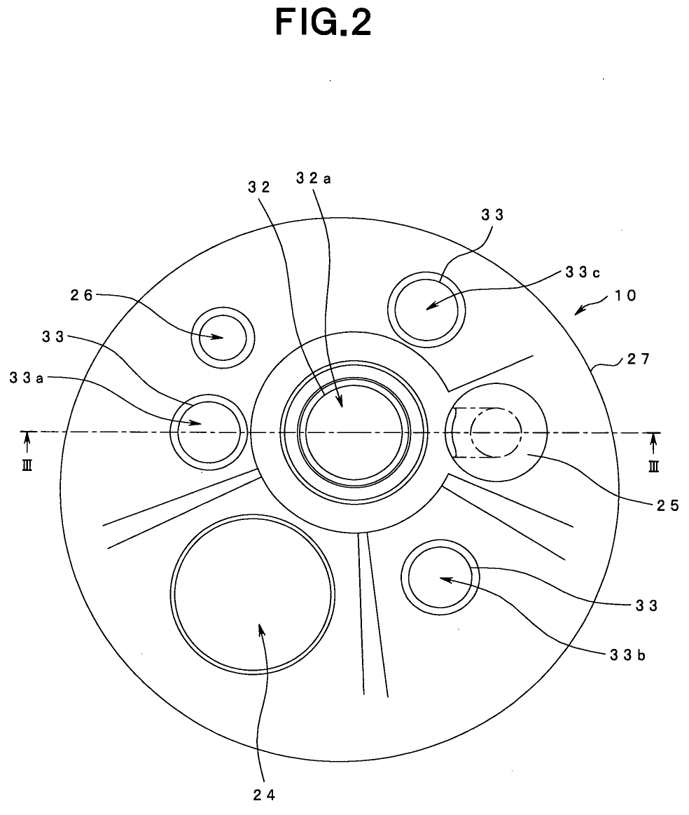

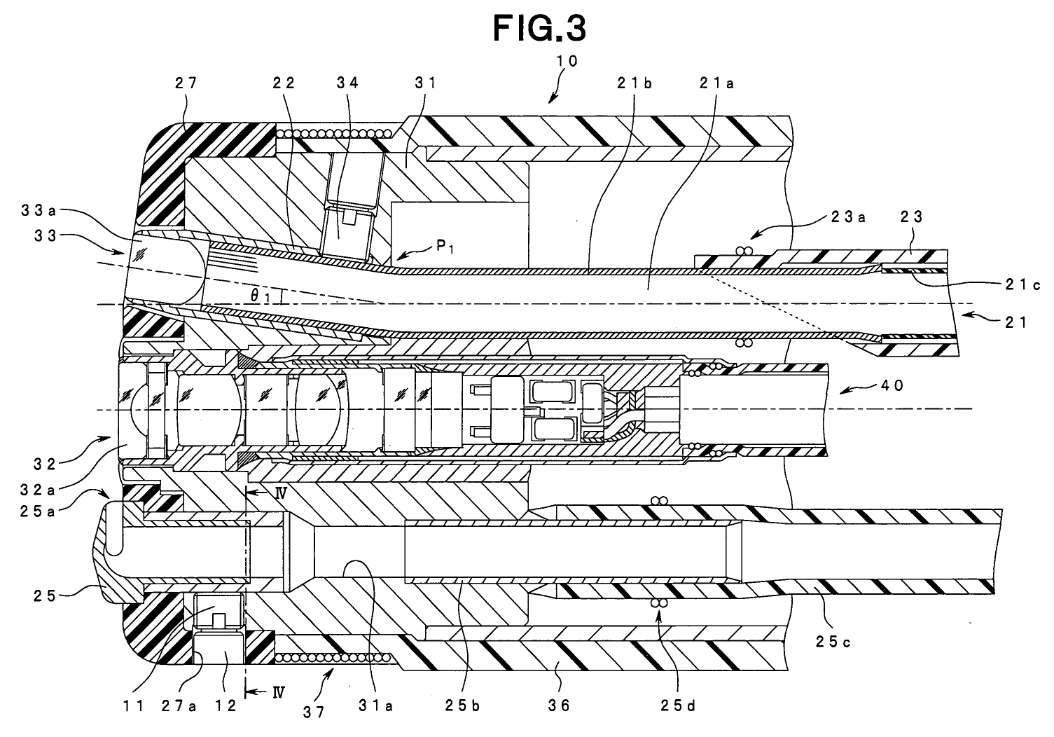

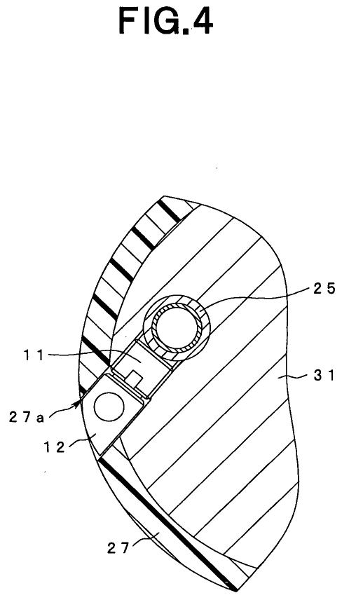

First of all, a structure of an embodiment apparatus will be described with reference to FIGS. 1 to 4. FIG. 1 is an explanatory diagram schematically showing an endoscope apparatus according to an embodiment of the invention. FIG. 2 is a plan view showing the distal surface of an insertion portion of the endoscope. FIG. 3 is a section diagram of the distal end section of the endoscope taken along the Line III-III in FIG. 2. FIG. 4 is a partial section diagram of the distal end section of the endoscope taken along the Line IV-IV in FIG. 3.

As shown in FIG. 1, the endoscope apparatus according to this embodiment includes an electronic endoscope (called “endoscope” hereinafter) 1, a light source device 5, a processor 6 and a monitor 7. The endoscope 1 includes an operation portion 2, an insertion portion 3 and a universal cord 3a. The operation portion 2 serves as operation means for controlling a bending operation and conduits. The insertion portion 3 serves as insertion means connected to the operation portion 2 on the proximal side for being inserted into a body cavity. The universal cord 3a extends from the operation portion 2 and has a connector portion 4 at the distal end. The connector portion 4 of the endoscope 1 is connected to the light source device 5 and the processor 6. The monitor 7 is connected to the processor 6.

The insertion portion 3 of the endoscope 1 has a flexible tube section 8, a bending section 9 and a distal end section 10. The flexible tube section 8 has flexibility. The bending section 9 is provided on the distal side of the flexible tube section 8. The distal end section 10 is provided on the distal side of the bending section 9. The distal surface of the distal end section 10 of the endoscope 1 according to this embodiment has a bullet form, and the distal end section 10 internally includes an image pickup unit, which will be described later.

The image pickup unit has an image pickup device such as a CCD and a CMOS for capturing an image of a part within a body cavity as described later. The image signal captured by the image pickup device is transmitted to the processor 6 through the universal cord 3a. The transmitted image signal undergoes signal processing in the processor 6, and an observation image 7a thereof is displayed on the monitor 7.

The operation portion 2 of the endoscope 1 has an operation knob that remotely bends the bending section 9 and a press button switch for an operation such as air feeding and water feeding.

The light source device 5 has an illumination light source and is connected to a light guide, which will be described later, through the connector portion 4. The light guide extends through the operation portion 2, insertion portion 3 and universal cord 3a. Furthermore, according to this embodiment, the light source device 5 includes an air/water-feeding source for feeding air and/or water to the conduit in the endoscope 1 and a suction source for suction from the conduit in the endoscope 1.

Next, the distal surface of the distal end section 10 and an internal structure of the distal end section 10 will be described with reference to FIGS. 2 and 3.

As shown in FIG. 2, the distal surface of the distal end section 10 has an observation lens 32a, illumination lenses 33a, 33b and 33c, a suction opening 24, an air/water feeding nozzle 25 and an opening 26. The observation lens 32a is provided within an observation window 32. The illumination lenses 33a, 33b and 33c are provided within respective three illumination windows 33, for example. The suction opening 24 also serves as an opening for a treatment tool. The air/water feeding nozzle 25 is for air/water feeding in order to clean a stain on the outer surface of the observation lens 32a. The opening 26 for a front water feeding nozzle is for cleaning blood and/or mucus of an affected part within a body cavity.

The suction opening 24, air/water feeding nozzle 25 and opening 26 for front water feeding are provided in respective areas among the illumination windows 33. More specifically, the suction opening 24 is in the area between the illumination lens 33a and the illumination lens 33b. The air/water feeding nozzle 25 is provided between the illumination lens 33b and the illumination lens 33c. The opening 26 for a front air/water feeding nozzle is in the area between the illumination lens 33a and the illumination lens 33c.

Next, an internal structure of the distal end section 10 will be described with reference to FIG. 3.

As shown in FIG. 3, the distal end section 10 internally includes a metallic distal rigid part 31. The distal rigid part 31 has multiple spaces for accommodating the image pickup unit 40 extending towards the proximal end of the observation window 32 and light guide units 21 provided on the respective proximal sides of the illumination windows 33. The distal rigid part 31 is covered by a distal cover 27, and the distal side of the distal rigid part 31 is covered by the distal cover 27.

The proximal part of the distal rigid part 31 is covered by a casing tube 36 of a synthetic resin or rubber, for example. The casing tube 36 is bonded and fixed to the distal rigid part 31 with threading 37.

The image pickup unit 40 has an observation optical system including an observation window lens and multiple lenses, a cover glass and an image pickup device such as a CCD and a CMOS. In this embodiment, the observation viewing angle with the image pickup unit 40 is defined as wide as about 140° to 170°.

Based on incident light through the observation window 32, the image signal captured by the image pickup device of the image pickup unit 40 is transmitted to the processor 6 (see FIG. 1). In this case, the processor 6 performs image processing on the received image signal and creates data of a substantially rectangular observation image 7a.

As shown in FIG. 1, the substantially rectangular observation image 7a is a rectangle with four corners cut, that is, so-called electronically masked corners and is displayed on the monitor 7 as an octagonal observation image. Notably, in this case, the optical system of the image pickup unit 40 is defined for preventing the air/water feeding nozzle 25 from coming into the observation field of view of the image pickup unit 40.

Each of the light guide units 21 has the illumination lens 33a and an optical fiber bundle 21a serving as a light guide. The distal part of the optical fiber bundle 21a is bonded and fixed within a metal pipe 22. The distal part of the optical fiber bundle 21a and the illumination lens 33a are inserted into and fixed within a cylindrical frame 22.

Each of the light guide units 21 is inserted into a space of the distal rigid part 31 and is fixed to the distal rigid part 31 together with the frame 22 by a fixing screw 34. The optical fiber bundle 21a on the proximal side than the metal pipe 21b is covered by a soft tube 21c. Furthermore, a part of the metal pipe 21b on the proximal side and the tube 21c are covered by the casing tube 23.

The casing tube 23 is bonded and fixed to the metal pipe 21b with threading 23a. Furthermore, in this embodiment, the metal pipe 21b is bent toward the outer circumference of the distal end section 10 at a predetermined angle θ1 at the middle position P1. The angle θ1 is an angle as much as about 8°, for example. As a result, the optical fiber bundle 21a extends within the metal pipe 21b along the bending form of the metal pipe 21b.

The light guide units 21 provided on the proximal side of the other illumination lenses 33b and 33c are also bent toward the outer circumference of the distal end section 10 at the predetermined angle 01 along the bending form of the metal pipe 21b on the distal side. Thus, the illumination range of the light guide units 21 is defined in accordance with the wide range of field of view of the image pickup unit 40. Notably, the endoscope 1 may adopt an illumination member such as an LED instead of the light guide units 21.

The air/water feeding nozzle 25 provided in the distal rigid part 31 has an opening 25a at the distal end part. The opening 25a directs such that water or air sprayed from the air/water feeding nozzle 25 can be supplied toward the outer surface of the observation lens 32a.

The proximal side of the air/water feeding nozzle 25 has a pipe form and is fixed with a fixing member 11 which serves as fixing means such as a screw screwed into a screw hole. The screw hole is a hole in the distal rigid part 31. The distal cover 27 covering the distal surface of the distal rigid part 31 has a hole 27a.

The hole 27a communicates with the screw hole in the distal rigid part 31 into which the fixing member 11 is screwed with the distal rigid part 31 covered by the distal cover 27. In other words, with the distal rigid part 31 covered by the distal cover 27, the fixing member 11 is inserted into the screw hole in the distal rigid part 31 from the hole 27a, and the air/water feeding nozzle 25 is fixed to the distal rigid part 31.

After the air/water feeding nozzle 25 is fixed to the distal rigid part 31 with the fixing member 11, a sealing pin 12 is inserted into the hole 27a of the distal cover 27 as shown in FIG. 4. The sealing pin 12 is an endoscope hole sealing part of the invention, which will be described later. The sealing pin 12 is fixed therein with an adhesive.

The distal rigid part 31 has a hole 31a, which is sealed means communicating with the air/water feeding nozzle 25. A connecting tube 25b is inserted into and fixed to the proximal side of the hole 31a. A water feeding tube 25c is connected to the proximal side of the connecting tube 25b.

Thus, a water/air feeding conduit comprises the air/water feeding nozzle 25, the hole 31a in the distal rigid part 31, the connecting tube 25b and the water feeding tube 25c. The water feeding tube 25c is bonded and fixed to the connecting tube 25b with threading 25d at the distal end part.

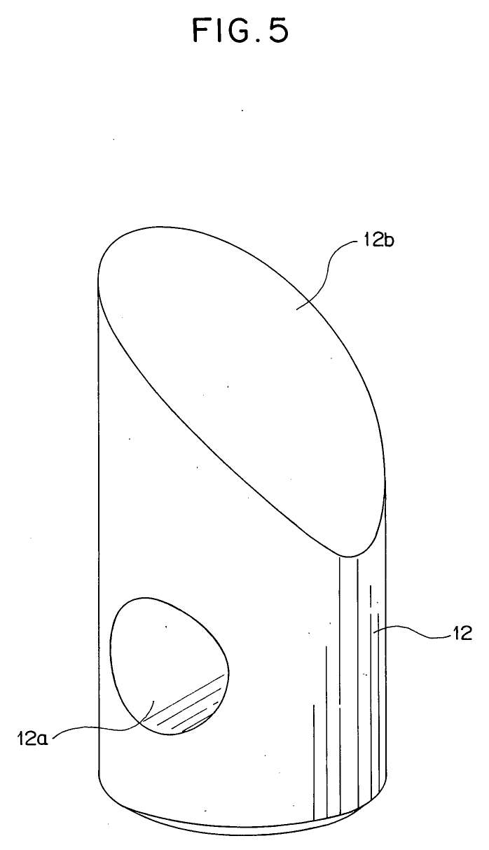

Next, the sealing pin 12, which is an endoscope hole sealing part of the invention, will be described with reference to FIGS. 5 to 9.

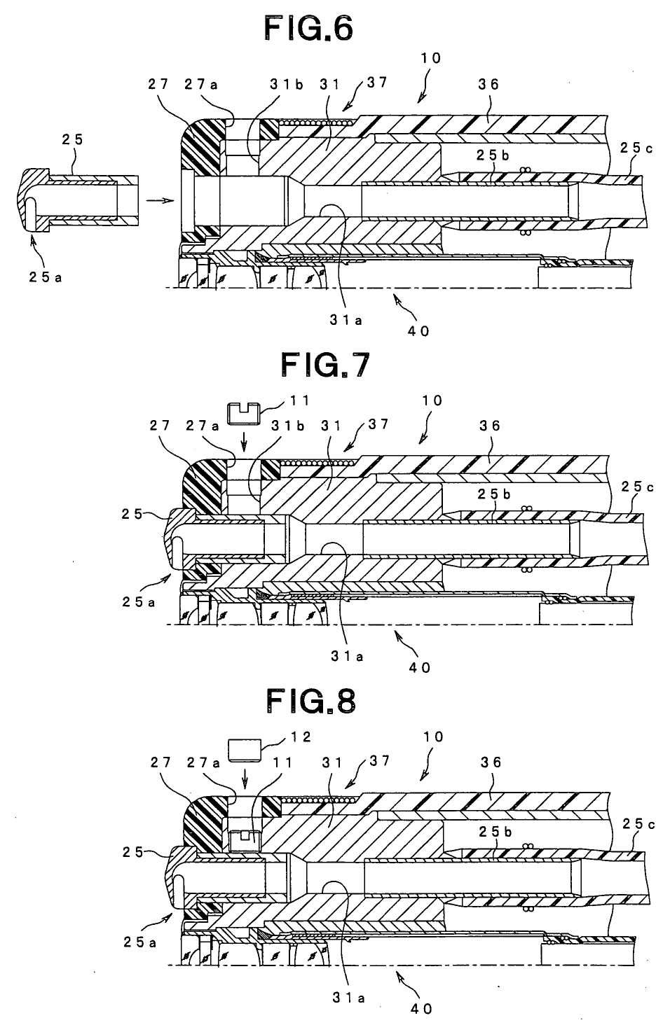

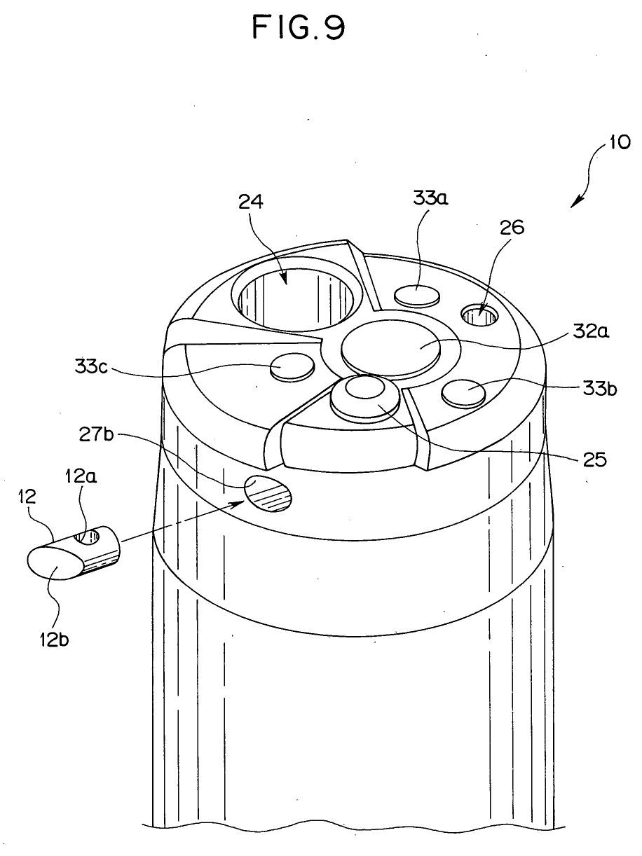

FIG. 5 is an appearance diagram of the sealing pin 12, which is an endoscope hole sealing part of the invention. FIG. 6 is a diagram for explaining assembly in which the air/water feeding nozzle 25 is inserted from the distal surface of the distal cover 27 to the distal rigid part 31. FIG. 7 is a diagram for explaining assembly in which the fixing member 11 is inserted from the hole 27a in the distal cover 27 to the distal rigid part 31. FIG. 8 is a diagram for explaining assembly in which the sealing pin 12 is inserted into the hole 27a in the distal cover 27. FIG. 9 is a perspective view of the distal end section 10 in the state shown in FIG. 8.

The sealing pin 12 of the invention shown in FIG. 5 has a body portion 12A, a hole 12a and an end face 12b. The body portion 12A serves as substantially columnar sealing means made of a synthetic resin having biological adaptability, such as a polysulfone resin and a polyether sulfone resin. The hole 12a is punched at the center of the body portion 12A in the direction substantially orthogonal to the axial direction. The end face 12b serves as outer surface forming means on one smooth curved surface of the body portion 12A.

The sealing pin 12 is defined to have a substantially equal longitudinal length to the length from the hole surface on the outer circumference of the hole 27a punched to the distal cover 27 to the surface of the fixing member 11 fixing the air/water feeding nozzle 25 within the screw hole in the distal rigid part 31. The surface curvature of the end face 12b of the sealing pin 12 is defined to have a curved surface form having an equal curvature to that of the hole surface of the hole 27a of the distal cover 27.

With the above arrangement, the sealing pin 12 of the present invention is fixed into the screw hole of the distal rigid part 31 and the hole 27a of the distal cover 27 with an adhesive such as a sealant and a filler, after the air/water feeding nozzle 25 is fixed with the fixing member 11. The sealing pin 12 is a part for defining the distal cover 27 in the hole 27a of the distal cover 27 to have a smooth curved side circumferential surface.

For example, since the air/water feeding nozzle 25 projects on the distal surface of the distal end section 10, the air/water feeding nozzle 25 may be hit by an operating table, for example, under some user's handling, which may crush the opening 25a. In this case, the air/water feeding nozzle 25 is replaced.

In replacing the air/water feeding nozzle 25, a repair stuff may destroy and remove the sealing pin 12 from the screw hole in the distal rigid part 31 and the hole 27a in the distal cover 27. Then, the repair stuff removes the fixing member 11 fixing the air/water feeding nozzle 25 to the distal rigid part 31 and replaces the air/water feeding nozzle 25.

The sealing pin 12 has the hollow hole 12a so that a needle, for example, can be hooked on the hole 12a for easy removal of the sealing pin 12 from the screw hole in the distal rigid part 31 and the hole 27a in the distal cover 27 in replacing the air/water feeding nozzle 25.

The hole 12a causes an unnecessary adhesive to flow into a space since the adhesive coated on the outer circumferential surface of the sealing pin 12 enters from the openings on both ends.

Next, a state in which the sealing pin 12 is assembled into the screw hole in the distal rigid part 31 and the hole 27a in the distal cover 27 will be described with reference to FIGS. 6 to 9.

First of all, the air/water feeding nozzle 25 is inserted, as shown in FIG. 6, from the opening in the distal surface of the distal cover 27 communicating with the hole 31a in the distal rigid part 31 from the end on the opposite side of the opening 25a.

Next, the fixing member 11 such as a screw is inserted from the hole 27a in the distal cover 27 as shown in FIG. 7 and is screwed into a hole 31b in the distal rigid part 31. One end face is pressed against the side circumferential surface of the air/water feeding nozzle 25 so that the air/water feeding nozzle 25 can be fixed to the distal rigid part 31.

Next, an adhesive is coated on the side circumferential surface of the body portion 12A, and the sealing pin 12 is inserted from the hole 27a of the distal cover 27 as shown in FIGS. 8 and 9. Here, the body portion 12A of the sealing pin 12 is fixed to the distal cover 27 and the distal rigid part 31 with the end face 12b placed within the same plane as the curved plane of the side circumferential surface of the distal cover 27, which is the outer surface of the endoscope.

In other words, as described above, the end face 12b provides the sealing pin 12 with a smooth curved surface form so as to prevent any level change in the side circumferential surface of the distal cover 27 in the hole 27a in the distal cover 27.

As a result, in the distal end section 10 of the endoscope 1, the hole 27a in the distal cover 27 is sealed with the sealing pin 12 of the invention so that the appearance of the side circumferential surface of the distal cover 27 can have a smooth curved surface form. Thus, the side circumferential surface of the distal end section 10 of the endoscope 1, that is, the side circumferential surface of the distal cover 27 in this case does not have any level change. Therefore, the insertion portion 3 of the endoscope 1 can have improved insertability into a body cavity of a subject and improved disinfectability after use.

Alternatively, a sealant or a filler, for example, may be used to fill the hole 27a in order to seal the hole 27a in the distal cover 27. However, the sealant or filler may have a sink mark in the curing process, or adjusting the amount to fill may be difficult, which lowers the workability. Therefore, it is greatly difficult that the outer surface of the side circumferential surface of the distal cover 27 has a smooth curved surface form only with a sealant or a filler, for example.

Accordingly, the adoption of the sealing pin 12 of the invention can easily provide the outer surface of the side circumferential surface of the distal cover 27 with a smooth curved surface form.

Furthermore, when the air/water feeding nozzle 25 must be replaced, a repair stuff can easily pull out the sealing pin 12 of the invention by using a needle, for example, and can remove the fixing member 11 fixing the air/water feeding nozzle 25. Thus, the workability for the replacement of the air/water feeding nozzle 25 can be improved. In other words, the repair stuff can easily replace the air/water feeding nozzle 25 without removing the distal cover 27 from the distal rigid part 31.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscope hole sealing part comprising:
a body portion which seals a hole into which a fixing member is inserted, the fixing member fixing a built-in part in an endoscope; and
an end face which is one surface of the body portion and is the outer surface of the endoscope.

2. The endoscope hole sealing part according to claim 1, wherein the end face has a smooth curved surface.

3. The endoscope hole sealing part according to claim 2, wherein the smooth curved surface has a curvature defined equal to the curvature of the side circumferential surface of the endoscope.

4. The endoscope hole sealing part according to claim 1, wherein the end face is within the same plane as that of the outer surface of the endoscope.

5. The endoscope hole sealing part according to claim 1, wherein the body portion has a hole in the direction substantially orthogonal to the longitudinal direction.

6. The endoscope hole sealing part according to claim 1, wherein the sealing part includes a member having biological adaptability.

7. The endoscope hole sealing part according to claim 6, wherein the member having biological adaptability is a synthetic resin.

8. The endoscope hole sealing part according to claim 1, wherein the body portion is fixed to the hole with an adhesive.

9. The endoscope hole sealing part according to claim 2, wherein the end face is within the same plane as that of the outer surface of the endoscope.

10. The endoscope hole sealing part according to claim 3, wherein the end face is within the same plane as that of the outer surface of the endoscope.

11. The endoscope hole sealing part according to claim 2, wherein the body portion has a hole in the direction substantially orthogonal to the longitudinal direction.

12. The endoscope hole sealing part according to claim 3, wherein the body portion has a hole in the direction substantially orthogonal to the longitudinal direction.

13. The endoscope hole sealing part according to claim 4, wherein the body portion has a hole in the direction substantially orthogonal to the longitudinal direction.

14. The endoscope hole sealing part according to claim 2, wherein the sealing part includes a member having biological adaptability.

15. The endoscope hole sealing part according to claim 14, wherein the member having biological adaptability is a synthetic resin.

16. The endoscope hole sealing part according to claim 3, wherein the sealing part includes a member having biological adaptability.

17. The endoscope hole sealing part according to claim 16, wherein the member having biological adaptability is a synthetic resin.

18. An endoscope hole sealing part in an endoscope including an insertion portion having a distal end section provided with an image pickup unit for being inserted into a body cavity, an operating portion connected to the proximal side of the insertion portion and an air/water feeding nozzle which communicates with an air/water feeding tube within the operation portion and the insertion portion and is fixed to the distal end section with a fixing member, the endoscope hole sealing part comprising:
a body portion which seals a hole at the distal end section into which the fixing member is inserted; and
an end face which is one surface of the body portion,
wherein the body portion is fixed to the hole in such a way that the end face can be within the same plane as the outer surface of the distal end section.

19. An endoscope hole sealing part comprising:
sealing means which seals sealed means into which fixing means is inserted, the fixing means fixes air/water feeding means in an endoscope; and
outer surface forming means which is one surface of the sealing means and forms the outer surface of the endoscope.

20. The endoscope hole sealing part according to claim 19, wherein the outer surface forming means is within the same plane as the outer surface of the endoscope.
